(19) United States
(12) Patent Application Publication
(10) Pub. No.: US 2015/0208691 A1
(43) Pub. Date: Jul. 30, 2015

(54) NOVEL USE OF ALKOXYLATES OF MONO- AND POLYVALENT ALCOHOLS

(71) Applicant: AB AGRI LIMITED, Marlborough Park, Wiltshire (GB)

(72) Inventors: Michael Bedford, Beckhampton (GB); Nicola Walker, Marlborough (GB)

(21) Appl. No.: 14/414,052

(22) PCT Filed: Jul. 12, 2013
(86) PCT No.: PCT/GB2013/000301

§ 371 Date: Jan. 9, 2015

(30) Foreign Application Priority Data
Jul. 12, 2012 (GB) 1212463.2

(51) Int. Cl. A23K 1/16; A23K 1/18; A61K 31/08

(52) U.S. Cl. CPC A23K 1/1609; A61K 31/08; A23K 1/1813

(57) ABSTRACT

The present invention relates to the use of certain non-ionic surfactants, in particular alkoxylates of mono- and poly-valent alcohols, as inhibitors of methane production and/or in reducing methanogen production in vitro and/or in vivo. Such compounds have been found to be particularly useful for methane mitigation in ruminants.

The present invention relates to the use of certain non-ionic surfactants, in particular alkoxylates of mono- and polyvalent alcohols, as inhibitors of methane production in ruminants and/or in reducing methanogen production in vitro and/or in vivo.

Methane production through enteric fermentation is of concern worldwide due to its contribution to the accumulation of greenhouse gases in the atmosphere. Greenhouse gases such as carbon dioxide, methane, nitrous oxide and ozone contribute to climate change and global warming through their absorption of infrared radiation in the atmosphere. Although classified as a trace gas, methane is recognised as the second largest anthropogenic greenhouse gas behind carbon dioxide and has a 12-year atmospheric lifetime. Globally, 50 to 60% of methane emissions are from the agricultural sector, specifically from livestock operations. Within this sector, the principal source of methane is from ruminant animals (S. E. Hook et al., “Methanogens: Methane Producers of the Rumen and Mitigation Strategies”, Archaea, Vol. 2010, Article ID 945785, 11 pages, 2010).

Methane is produced in the rumen as a product of normal fermentation of feedstuffs. Digestion of plant material and food in the rumen occurs due to the combined action of microbial fermentation and physical breakdown during rumination. The microbes involved include bacteria, protozoa and anaerobic fungi. Although the major end point of fermentation is the formation of hydrogen and carbon dioxide, hydrogen does not accumulate in the rumen. Methanogens and other hydrogen-utilising bacteria readily utilise any hydrogen produced during fermentation and in so doing help to maintain a low partial pressure of hydrogen which is necessary to ensure optimal fermentation and degradation of plant cell walls. If hydrogen is accumulated, it can impact negatively on fermentation. Although methane production can also occur in the lower GI tract, as in non-ruminants, ca. 90% of methane emitted from ruminants is produced in the rumen and exhaled through the mouth and nose. As methane is exhaled into the atmosphere, the ruminant suffers loss of ingested feed-derived energy and a reduction in feed efficiency.

In view of the foregoing, there exists a need for the development of novel methane reduction strategies in ruminants. Known strategies have focused upon one of two general approaches: (1) implementing a direct effect on methanogens (i.e. methane-producing bacteria); or (2) implementing an indirect effect on methanogens by altering substrate availability for methanogenesis, for example via an effect on other rumen microbes.

The use of certain food supplements, including lipids, fatty acids and oils (such as palm or coconut oil), has previously been shown to reduce methane production in certain ruminants. However, the administration of such supplements can undesirably lead to a reduction in milk production, due to reduced fibre digestion, which is disadvantageous from a commercial agricultural perspective. Allicin, an organosulfur compound derived from garlic plants, has been shown to be effective at reducing methane emissions by 20% in sheep, but was not as effective in cows and also tainted and flavoured the milk.

The present invention is based upon the unexpected finding that certain non-ionic surfactants, in particular alkoxylates of mono- and polyvalent alcohols, may act as inhibitors of methane production in ruminants. Advantageously, the non-ionic surfactants of the present invention have been shown to exhibit no adverse effects on milk production, rumen pH, or rumen fermentation in vitro and in vivo.

According to a first aspect of the present invention there is provided an alkoxylate of a mono- or polyvalent alcohol, or a mixture thereof, for use as a methane mitigator and/or an inhibitor of methane production in ruminants. This effect is believed to occur due to a reduction in the number of methanogenic bacteria and by altering rumen fermentation, leading to an increase in propionate production.

According to a second aspect of the invention, there is provided a method of inhibiting methane production in ruminants, which method comprises administering an alkoxylate of a mono- or polyvalent alcohol, or a mixture thereof, to a ruminant in need thereof.

According to a third aspect of the invention, there is provided use of an alkoxylate of a mono- or polyvalent alcohol, or a mixture thereof, in inhibiting methane production in ruminants

According to a fourth aspect of the present invention, there is provided a composition comprising an alkoxylate of a mono- or polyvalent alcohol, or a mixture thereof, for use as an inhibitor of methane production in ruminants.

According to a fifth aspect of the present invention, there is provided a ruminant feed comprising an alkoxylate of a mono- or polyvalent alcohol, or a mixture thereof; preferably for use as an inhibitor of methane production.

According to a sixth aspect of the present invention, there is provided a method of reducing methanogen production in vitro and/or in vivo, which method comprises administering an alkoxylate of a mono- or polyvalent alcohol, or a mixture thereof.

According to a seventh aspect of the present invention there is provided an alkoxylate of a mono- or polyvalent alcohol, or a mixture thereof, for use in reducing methanogen production in vitro and/or in vivo.

According to an eighth aspect of the invention, there is provided use of an alkoxylate of a mono- or polyvalent alcohol, or a mixture thereof, in reducing methanogen production in vitro and/or in vivo.

According to a ninth aspect of the present invention, there is provided a composition comprising an alkoxylate of a mono- or polyvalent alcohol, or a mixture thereof, for use in reducing methanogen production in vitro and/or in vivo.

According to a tenth aspect of the present invention, there is provided a ruminant feed comprising an alkoxylate of a mono- or polyvalent alcohol, or a mixture thereof; preferably for use in reducing methanogen production in vitro and/or in vivo.

The term “alkoxylate of a mono- or polyvalent alcohol” as used herein refers a mono- or polyvalent alcohol comprising one or more alkylene oxide groups.

The alcohol group may be a primary, secondary or tertiary alcohol, but is preferably a primary or secondary alcohol. The alcohol group preferably comprises a linear or branched C₈₋₂₄ alkyl group, and more preferably a linear or branched C₈₋₁₈ alkyl group. In a preferred embodiment of the invention, the alcohol is monovalent. In a further preferred embodiment of the invention, the alcohol comprises one or more ethylene oxide groups, such as from 3 to 15 ethylene oxide groups. Examples of suitable compounds include commercially available non-ionic surfactants classified as “alcohol ethoxylates”.

Preferred alkoxylates of a mono- and/or polyvalent alcohols for use in the present invention include ethoxylated compounds of formula (I), and mixtures thereof,

R(OC₂H₄)ₙ(OH) (I)

wherein,

R represents a linear or branched C₈₋₂₄ alkyl group; and

n represents an integer from 3 to 15;

Preferably, R represents a linear or branched C₈₋₁₈ alkyl group; and more preferably a linear or branched C₁₂₋₁₅ alkyl group, or mixtures thereof.

Preferably, the alkoxylate of a mono- and/or polyvalent alcohol is a C₁₂-C₁₅ ethoxylate alcohol.

Preferably, n represents an integer from 3 to 12, more preferably from 5 to 12, for example 5, 6, 7, 8, 9, 10, 11 or 12, most preferably 7.

A particularly preferred example of a commercially available alcohol ethoxylate is Surfac LM70/90 (a C₁₂-C₁₅ alcohol ethoxylated with 7 moles of ethylene oxide). Surfac AC LM 70/90 is commercially available from Surfachem Group Ltd.

Alkoxylates of mono- or polyvalent alcohols for use in the present invention are commercially available or may be prepared by conventional methods known in the art. By way of example, compounds of formula (I) may be prepared by reaction of a suitable linear or branched alcohol (II) with ethylene oxide (III) in the presence of a suitable basic catalyst (such as sodium or potassium hydroxide) as follows:

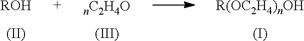

where n is defined with reference to formula (I) above.

It will be appreciated that the degree of alkoxylation (n) is a factor in determining the surfactant properties of the resulting compound, including the hydrophilic-lipophilic balance (HLB) thereof.

Non-ionic surfactants of the type described above may be administered to ruminants to inhibit methane production and/or to reduce methanogen production in vitro and/or in vivo; preferably, they are administered to inhibit methane production from enteric rumen fermentation.

The non-ionic surfactants described above may be used to inhibit methane production and/or to reduce methanogen production in vitro and/or in vivo in any ruminant species, including, but not limited to, cattle, sheep, goats, buffalo, antelope, bison, deer, elk, giraffes and camels; preferably cattle, sheep and goats; most preferably cattle.

Suitable modes of administration include drenching (i.e. administration via a cannula or other suitable delivery means, direct to the rumen) and/or administering the non-ionic surfactant with feed. The non-ionic surfactant may be administered alone or as a composition (such as a concentrate) comprising one or more additional active and/or non-active ingredients.

Examples of suitable active ingredients which may be co-administered with the above-mentioned non-ionic surfactants in accordance with the present invention include one or more of the following: garlic extracts (for example Garlic G-Pro nature and Garlic Allicin), plant extracts (for example essential oils, tannins and saponins), yeast cultures, antibiotics, bacteriocins, probiotics, ionophores (for example monensin), oils (for example coconut oil, palm kernel oil, linseed oil, soy oil and sunflower oil), fatty acids (for example lauric acid and myrstic acid), enzymes (for example, cellulases and hemicellullases), organic acids (for example, acrylic acid, citric acid, fumaric acid, malic acid and succinic acid), methanogenic inhibitors (for example, 2-bromoethanesulphonate, propynoic acid, nitroethane, ethyl trans-2-butenoate, 2-nitroethanol, sodium nitrate and ethyl-2-butynote) and other non-ionic surfactants (for example, alkyl polyglycoside, sorbitan trioleate and polyoxyethylene sorbitan monostearate).

Examples of suitable non-active ingredients which may be co-administered with the above-mentioned non-ionic surfactants in accordance with the present invention include, but are not limited to, water, plant oils and the like.

When administered with one or more other active ingredients, the non-ionic surfactants of the present invention may be administered simultaneously, separately or sequentially therewith. When the active ingredients are administered sequentially, either the non-ionic surfactant or the other active ingredient(s) may be administered first. When administration is simultaneous, the active ingredients may be administered either in the same or different compositions.

In one embodiment of the invention, there is provided a non-ionic surfactant of the type described above for use as a dietary supplement to inhibit methane production and/or to reduce methanogen production in vitro and/or in vivo in ruminants. In an alternative embodiment of the invention, there is provided a ruminant feed comprising a non-ionic surfactant of the type described above. Suitable feeds may be prepared by admixing a non-ionic surfactant with one or more carriers or diluents; such as, for example, maize silage, grass, forage plants, seeds, grains and cereals or mixtures thereof.

A suitable dosage range for administration of the above-mentioned non-ionic surfactants is from about 0.1 to, about 62.5 mg; preferably from about 0.5 to 50 mg, such as 0.5, 1, 2, 3, 4, 5, 10, 20, 30, 40 or 50 mg surfactant/g of dry matter (DM) of the feed. Most preferably, the dose is greater than 3 mg surfactant/g of dry matter of the feed or greater than 4 mg surfactant/g of dry matter of the feed.

The following non-limiting Examples are illustrative of the present invention. The experimental methods used in the Examples are based upon the following literature methods:

Cadillo-Quiroz H, Bräuer S, Yashiro E, Sun C, Yavitt J and Zinder S. Vertical profiles of methanogenesis and methanogens in two contrasting acidic peatlands in central New York State, USA. Environ Microbiol. 2006. 8(8):1428-1440.

Holben W E, Williams P, Saarinen M, Särkilahti L K and Apajalahti J H A. Phylogenetic Analysis of Intestinal Microflora Indicates a Novel Mycoplasma Phylotype in Farmed and Wild Salmon. Microbial Ecology. 2002 44: 175-185.

McDougall E. Studies on ruminant saliva. 1. The composition of output of sheep's saliva. Biochem J. 1948. 43:99-109.

Nadkarni M A, Martin F E, Jacques N A and Hunter N. Determination of bacterial load by real-time PCR using a broad-range (universal) probe and primers set. Microbiology. 2002. 148:257-266.Yu, Z and M. Morrison. 2004. Improved extraction of PCR-quality community DNA from digesta and fecal samples. Biotechniques 36:808-812.

### EXAMPLE 1

### Determining the Dose Response of an Alcohol Ethoxylate, Surfac LM70/90, on Rumen Fermentation in Vitro

The aim of this experiment was to determine the dose response of Surfac LM70/90 on rumen fermentation.

Eight different dosages of the alcohol ethoxylate (AE) were tested ranging from 0.5 to 50 mg. Samples were added to serum bottles containing 4 ml of rumen contents, 35 ml anaerobic McDougall's artificial saliva buffer and 1 g dried and chopped maize silage under CO₂, and then incubated at 39° C. for 24 h.

Total gas production was determined by positive volume displacement using a glass syringe. Gas samples were then analysed for, methane production; all the gas produced during the 24 hours was individually collected from the simulation vessels into evacuated 2 litre infusion bottles, which had ethane pre-introduced as an internal standard. The analyses were performed by gas chromatography using a flame ionisation detector for methane and ethane. Short chain volatile fatty acid (scVFA) production, DNA was isolated from samples collected from either the in vitro or in vivo studies using the bead beating and column clean-up method of Yu and Morrison (2004). Total bacteria were quantified using qPCR and specific primer sets and conditions as outlined in Nadkarni et al, 2002; total methanogens were quantified by qPCR using the primer sets and conditions as outlined by Cadillo-Quiroz et al, 2006. Specific primer sets and conditions for quantifying total protozoa were based on the method of Sylvester et al (Sylvester J T, Karnati S K, Yu Z, Morrison M and Firkins J L. Development of an assay to quantify rumen ciliate protozoal biomass in cows using real-time PCR. J Nutr. 2004. 134(12):3378-3384.)

Results

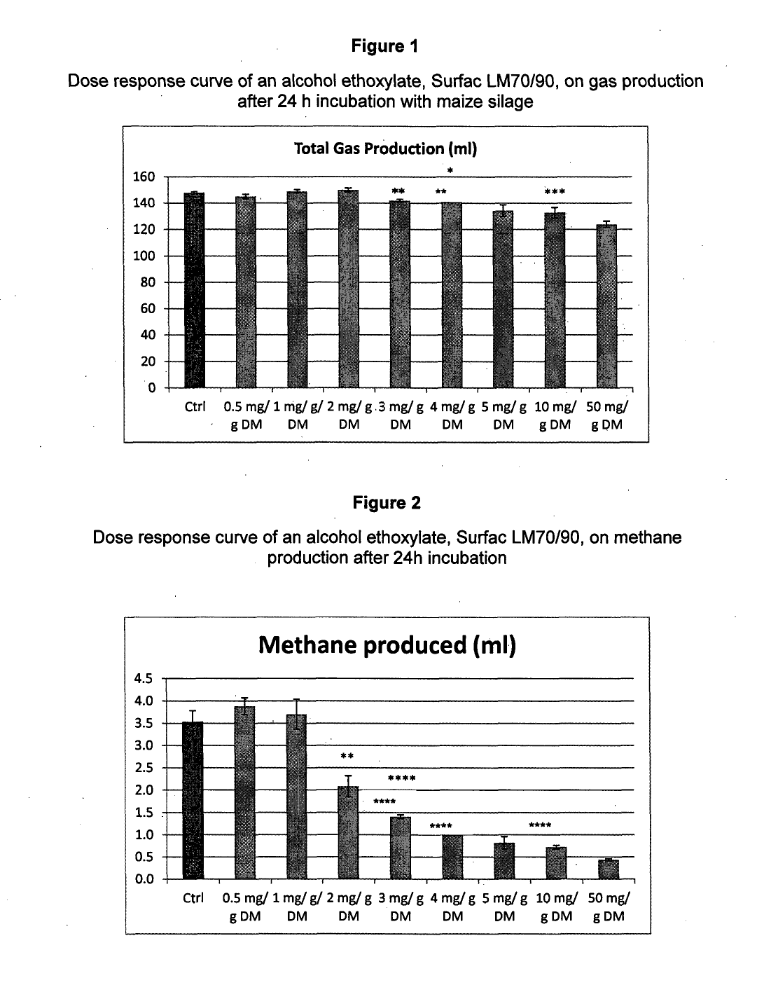

The results obtained are illustrated in FIGS. 1 to 10.

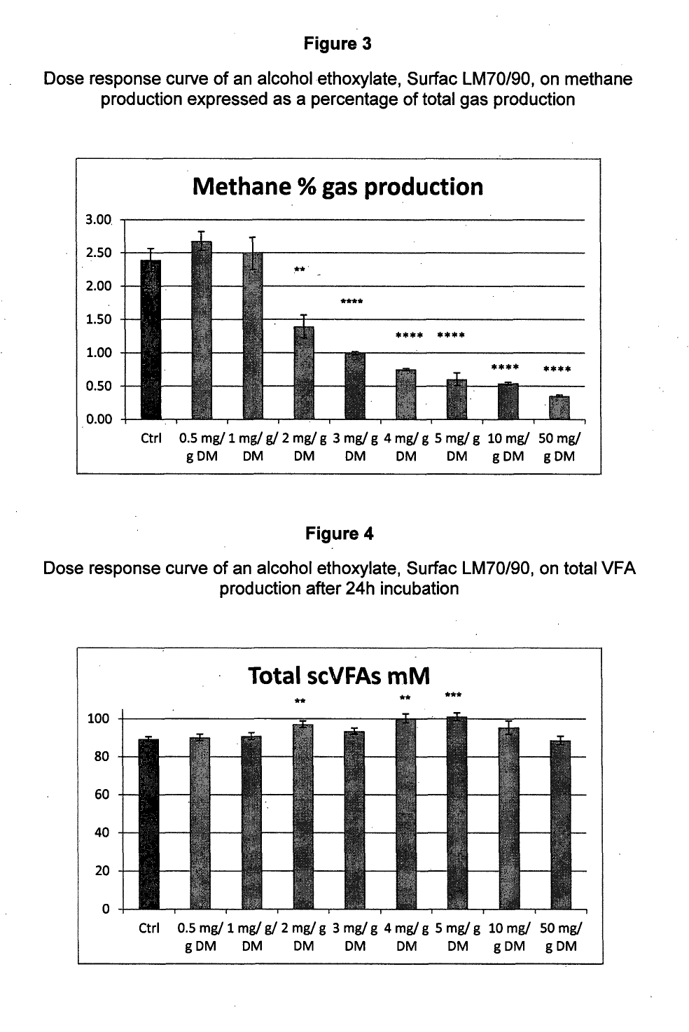

When the AE was added to the incubation, there was a significant reduction in gas production at a dose rate>3 mg/1 g dry matter (DM) maize silage (FIG. 1). This reduction in gas production was due to a reduction in methane formation (FIG. 2). At a dose rate>3 mg/g DM, methane production (ml) was reduced by 50% both as a direct measurement and when expressed as a percentage of the total gas production (FIG. 3).

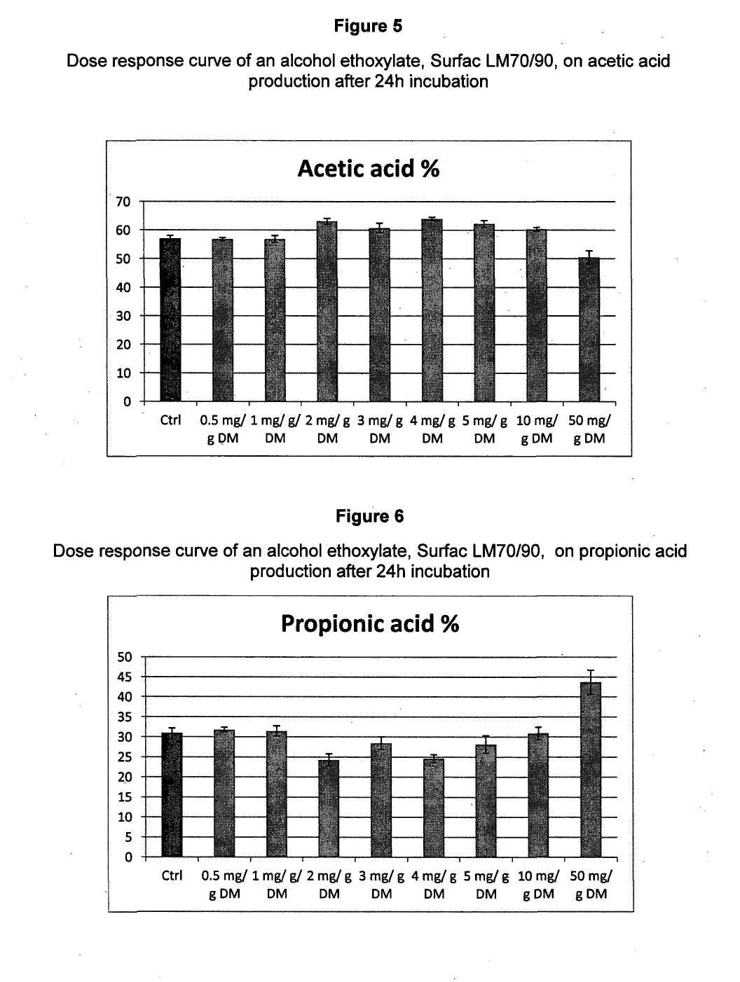

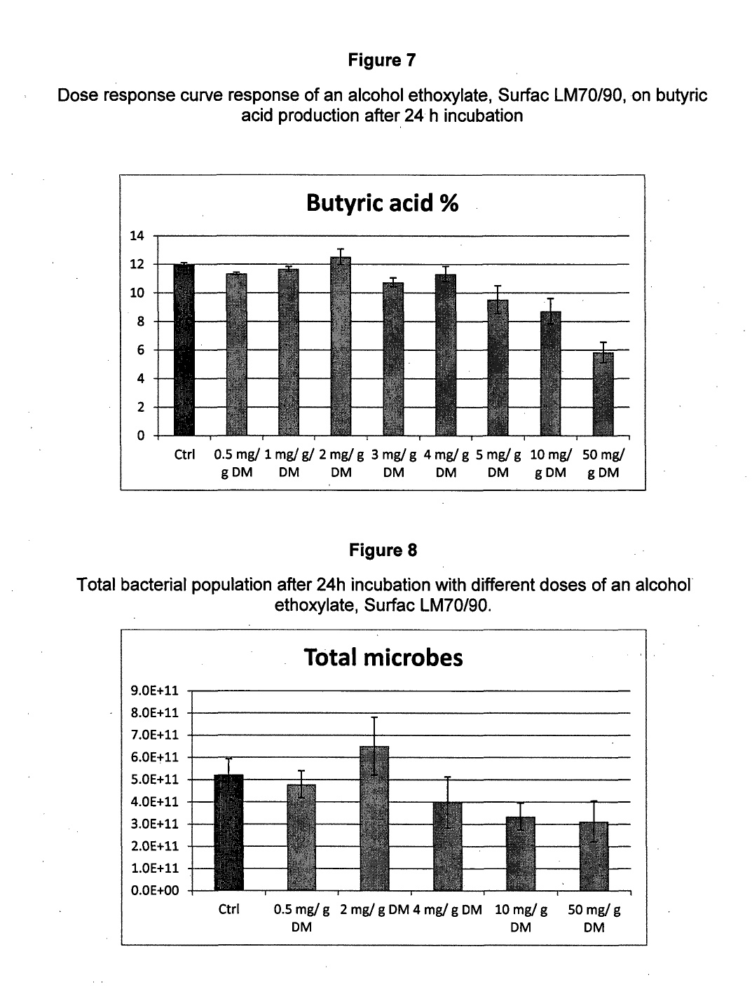

One problem which may be observed when methane is reduced is that rumen fermentation is also reduced. However, the AE unexpectedly stimulated rumen fermentation when measured as total VFA production when added at a dose rate between 2-10 mg/g DM (FIG. 4). At higher levels, the AE inhibited VFA production. The increase in total VFA production was mainly due to an increase in acetic acid production (FIG. 5). At the highest dose of AE, propionic acid was increased (FIG. 6) but butyric acid was decreased (FIG. 7).

Analysis of the total bacterial population revealed that although numerically there was a decrease in the bacterial population (FIG. 8), this reduction was not significant.

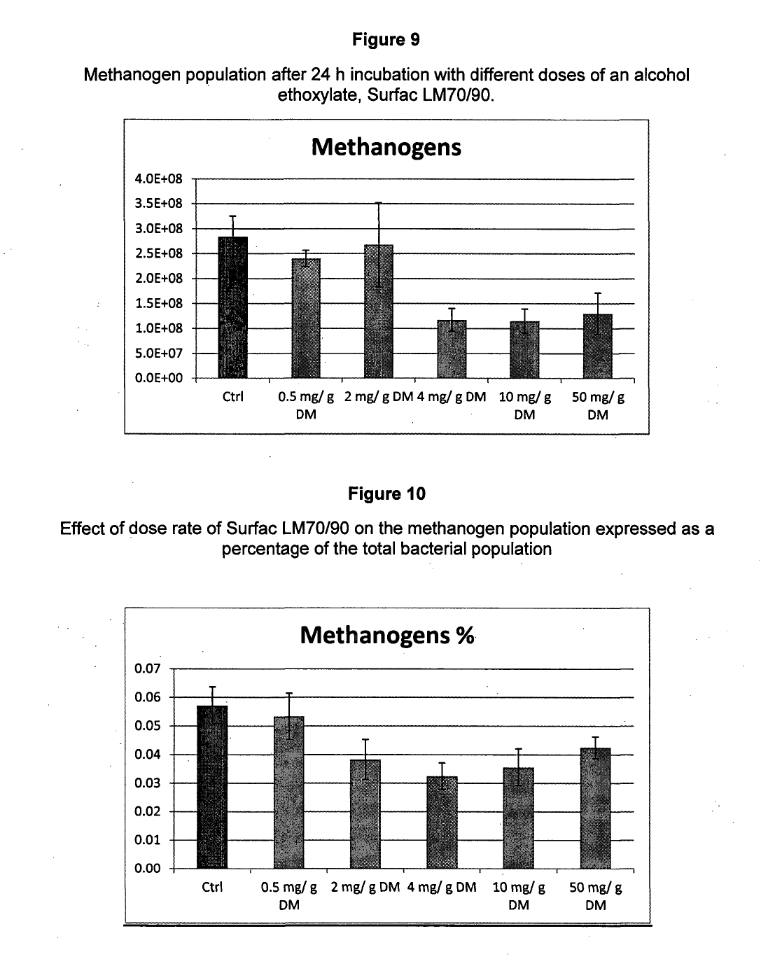

However, the decrease in the methanogenic population (FIG. 9) was significant, with methanogens being decreased by 50% at dose rates>4 mg/g DM.

### CONCLUSIONS

The addition of an AE at a concentration of 4 mg/g DM reduced the methanogenic population by 50% and at a dose rate greater than 3 mg/g DM, reduced methane production by 50%. Only when the AE was added at a concentration greater than 10 mg/g DM, was there a reduction in rumen fermentation and a decrease in VFA production. A shift in the rumen fermentation profile was also observed with an increase in acetic acid and propionic acid. Butyric acid production was decreased.

### EXAMPLE 2

### Effect of Different Doses of an Alcohol Ethoxylate, Surfac LM70/90, on Rumen Microbial Populations in Vivo

The aims of this experiment were to determine (i) whether the effects observed in Example 1 were also observed in vivo and (ii) whether an AE had a negative impact on milk production.

An experiment was set up using four ruminally fistulated lactating dairy cows fed a concentrate/hay diet. Measurements were taken during a 1 week control period, animals were then treated with a high dose of AE for 12 days (64 g/16 kg dry matter intake), then allowed to recover for 12 days, then subjected to further addition of a low dose of AE for 12 days (6.4 g/16 kg dry matter intake) followed by a further 12 day recovery period. Milk production was measured throughout the trial, rumen pH was measured hourly after feeding for a total of 8 hours on 2 different days, rumen samples were analysed for VFA content and rumen bacteria, protozoa, methanogens and populations of key bacterial species were enumerated using qPCR as outlined above. In addition to using qPCR to quantify the effect on total bacteria and methanogens, total protozoal populations were also enumerated using the specific primer set and method according to Sylvester et al, 2004.

Results

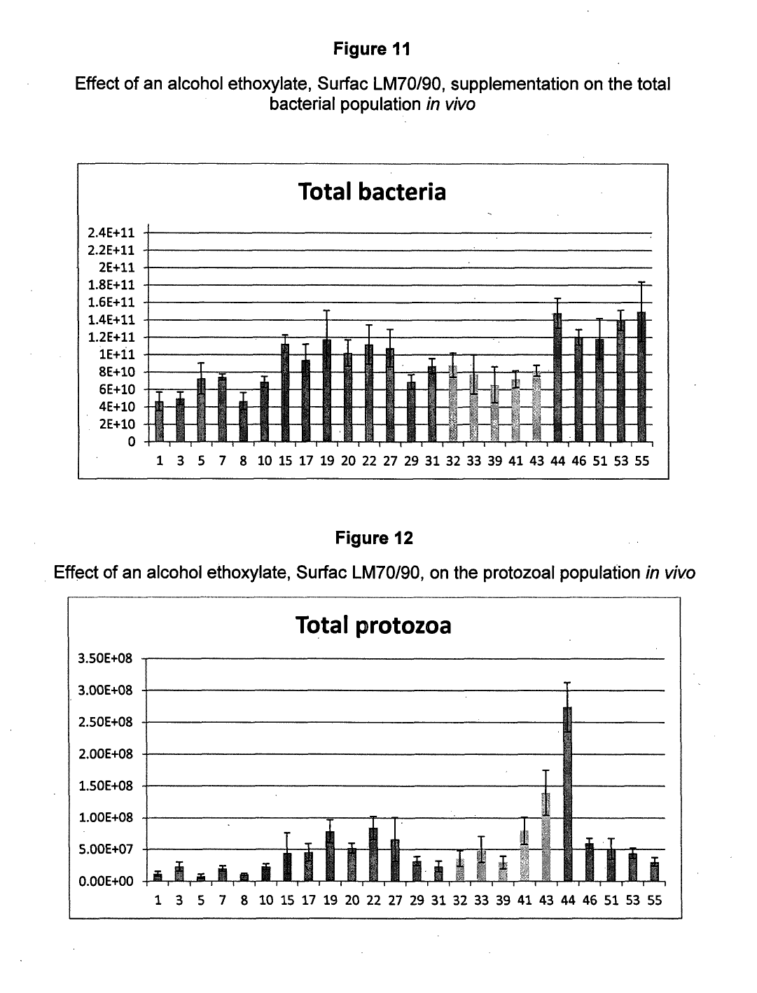

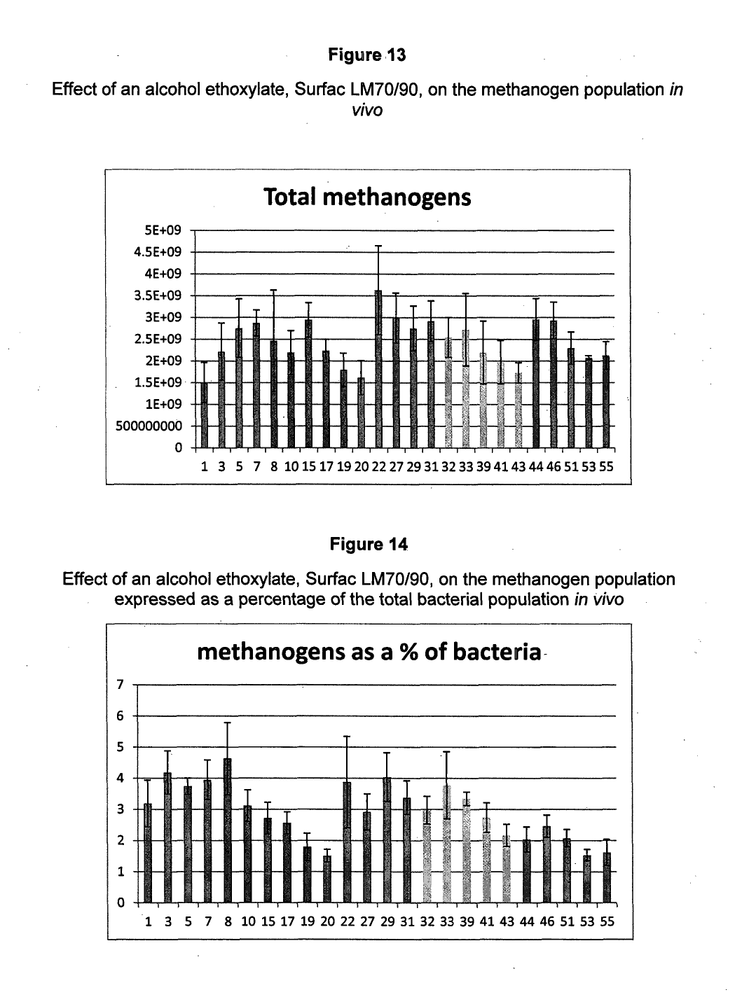

The results obtained are illustrated in FIGS. 11 to 14.

There was no significant effect on milk production or rumen pH (data not shown). The main effects were observed on the rumen flora. The total bacterial population was significantly increased during the period of AE application at the highest dose. During the washout period, the population then decreased. There was not such a significant increase in the total bacterial population at the low dose of AE, however, the population then increased in the washout period, indicating that there had been some effect with the AE which had been carried over.

Surprisingly, the addition of AE to the diet stimulated the protozoal population. Normally, most methane inhibitors work by reducing the protozoal population and disrupting the symbiotic relationship between the methanogens and the protozoa. As previously demonstrated in vitro, the addition of AE at the highest dose led to a significant reduction in the methanogen population especially when expressed as a percentage of the total bacterial population. As seen in vitro, the methanogens were reduced by approximately 50%.

### CONCLUSIONS

The addition of an AE to the diet resulted in a significant decrease in the methanogenic population. Although methane production from the animal was not measured in this instance, it can be assumed that this reduction would have closely correlated with a decrease in methane production. No negative effects on animal performance, rumen pH or rumen fermentation were observed during the experiment. This is unusual, as with the majority of methane inhibitors, there is generally an adverse effect on rumen fermentation as a result of impaired rumen function or fibre digestion.

1.-18. (canceled)

19. A method of reducing methanogen production in vitro and/or in vivo, which method comprises administering a composition comprising an alkoxylate of a mono- or polyvalent alcohol, or a mixture thereof, wherein the alkoxylate of a mono- or polyvalent alcohol is a compound of formula (I),
R(OC₂H₄)ₙ(OH) (I)
wherein,
R represents a linear or branched C₈₋₂₄ alkyl group; and
n represents an integer from 3 to 15.

20. The method according to claim 19, wherein R represents a linear or branched C₈₋₁₈ alkyl group, or a mixture thereof.

21. The method according to claim 19, wherein n represents an integer from 5 to 12.

22. The method according to claim 19, wherein the alkoxylate of a mono- or polyvalent alcohol is a C₁₂₋₁₅ ethoxylate alcohol.

23. The method according to claim 19, wherein the alkoxylate of a mono- or polyvalent alcohol is a C₁₂₋₁₅ alcohol ethoxylated with 7 moles of ethylene oxide.

24-41. (canceled)

42. A method of inhibiting methane production in ruminants, which method comprises administering a composition comprising an alkoxylate of a mono- or polyvalent alcohol, or a mixture thereof, wherein the alkoxylate of a mono- or polyvalent alcohol is a compound of formula (I),
R(OC₂H₄)ₙ(OH) (I)
wherein,
R represents a linear or branched C₈₋₂₄ alkyl group; and
n represents an integer from 3 to 15.

43. A method according to claim 42, wherein R represents a linear or branched C₈₋₁₈ alkyl group, or a mixture thereof.

44. A method according to claim 42, wherein the alkoxylate of a mono- or polyvalent alcohol is a C₁₂₋₁₅ ethoxylate alcohol.

45. A method according to claim 42, wherein n represents an integer from 5 to 12.

46. A method according to claim 42, wherein the alkoxylate of a mono- or polyvalent alcohol is a C₁₂₋₁₅ alcohol ethoxylated with 7 moles of ethylene oxide.

47-53. (canceled)

54. The method according to claim 61, wherein the ruminant feed comprises at least one member selected from the group consisting of maize silage, grass, forage plants, seeds, grains, and cereals.

55. The method according to claim 54, wherein the ruminant feed comprises maize silage.

56. The method according to claim 19, wherein the composition further comprises at least one member selected from the group consisting of a garlic extract, a plant extract, a yeast culture, an antibiotic, a bacteriocin, a probiotic, an ionophore, an oil, a fatty acid, an organic acid, a methanogenic inhibitor and a non-ionic surfactant.

57. The method according to claim 19, wherein the composition further comprises a ruminant feed.

58. The method according to claim 57, wherein the ruminant feed comprises at least one member selected from the group consisting of maize silage, grass, forage plants, seeds, grains and cereals.

59. The method according to claim 58, wherein the ruminant feed comprises maize silage.

60. The method according to claim 42, wherein the composition further comprises at least one member selected from the group consisting of a garlic extract, a plant extract, a yeast culture, an antibiotic, a bacteriocin, a probiotic, an ionophore, an oil, a fatty acid, an organic acid, a methanogenic inhibitor and a non-ionic surfactant.

61. The method according to claim 42, wherein the composition further comprises a ruminant feed.
